(19) United States
(12) Patent Application Publication
(10) Pub. No.: US 2008/0114340 A1
(43) Pub. Date: May 15, 2008

(54) SYSTEM AND METHOD FOR REDUCING PHOTON SCATTER IN DERMAL TISSUE

(75) Inventors: Matthew Fox, Bellaire, TX (US); Ashok Gowda, Houston, TX (US); Roger McNichols, Pearland, TX (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: BIOTEX, INC., Houston, TX (US)

(21) Appl. No.: 11/555,562

(22) Filed: Nov. 1, 2006

(51) Int. Cl. A61B 18/18

(52) U.S. Cl. 606/9

(57) ABSTRACT

A device includes an outer layer formed of a fluid impermeable polymer film. The outer layer forms an outer major surface of the device. The device also includes a reinforcement layer underlying the outer layer and an adhesive underlying the outer layer. The adhesive forms an annular adhesive surface wherein the device is configured to form a cavity between an inner surface of the device and a dermal surface when the annular adhesive surface is attached to the dermal surface. The device further includes a fluid conduit operable to provide fluid access to the cavity through the outer layer and the reinforcement layer.

### FIELD OF THE DISCLOSURE

This disclosure, in general, relates to systems and methods for reducing photon scatter in dermal or other tissues

### BACKGROUND

Irradiative methods and in particular, laser treatments are being used for a variety of dermatological treatments, including tattoo removal, hair removal, and treatment of ectatic vasculature. Particular wavelengths of lasers, for example, may be used to selectively agitate tissue to produce a desired effect. For example, laser treatment may be used to selectively degrade hair follicles or vascular tissue. In addition, laser treatments may be used to selectively degrade tissue storing ink pigments, resulting in release of pigment and ultimately removal of the pigment.

In particular, tattoos are created when particulate ink is forced through perforations in the skin. Much of the ink is carried away, but a portion is phagocytosed by dermal fibroblasts and can remain permanently. In most tattoos, the pigment-containing cells reside in the papillary or superficial dermis near the dermal-epidermal junction.

A dramatic increase in the number of people acquiring tattoos also has lead to a substantial increase in the demand for tattoo-removal. Typically, dermatological laser techniques are currently used. However, absorption and scattering by skin components superficial to the tattoo leads to limitations on the fluences and laser wavelengths that can be used for treatment. In particular, absorption and scattering may limit the amount of photons that reach the ink particulate, reducing the effectiveness of the treatment. Such reductions generally lead to reductions in the effective outcome of any one treatment, and often result in re-treatment of the same area. In addition, limitations placed on the selection of laser by the scattering of photons by the tissue results in a reduction in the effectiveness of treatment of particular, ink colors.

As such, an improved treatment system would be desirable.

### SUMMARY

In a particular embodiment, a device includes an outer layer formed of a fluid impermeable polymer film. The outer layer forms an outer major surface of the device. The device also includes a reinforcement layer underlying the outer layer and an adhesive underlying the outer layer. The adhesive forms an annular adhesive surface wherein the device is configured to form a cavity between an inner surface of the device and a dermal surface when the annular adhesive surface is attached to the dermal surface. The device further includes a fluid conduit operable to provide fluid access to the cavity through the outer layer and the reinforcement layer.

In another exemplary embodiment, a device includes a fluid impermeable film, a reinforcement material underlying the fluid impermeable film, and an adhesive forming at least an annular adhesive surface. The device is configured to form a cavity between an inner surface of the device and a dermal surface when the annular adhesive surface is attached to the dermal surface. The device also includes a fluid conduit configured to provide fluid access to the cavity through the fluid impermeable film and the reinforcement material.

In a further exemplary embodiment, a device includes a fluid impermeable film having an outer major surface and an inner major surface. The inner major surface includes an adhesive. The device also includes a reinforcement material underlying the fluid impermeable film. The reinforcement material has a elastic modulus greater than the elastic modulus of the fluid impermeable film. The device further includes a liner underlying the reinforcement material. An outer contour of the liner extends beyond an outer contour of the reinforcement material. An outer contour of the fluid impermeable film extends beyond the outer contour of the liner. The liner includes an adhesive surface adhered to the reinforcement material and a portion of the fluid impermeable film and a non-adhesive surface configured to form a cavity between a dermal surface and the liner when the device engages the dermal surface.

In an additional embodiment, a kit includes a dermal clearing agent and a dermal treatment devices. The dermal treatment device includes an outer layer formed of a fluid impermeable polymer film. The outer layer forms an outer major surface of the dermal treatment device. The device further includes a reinforcement layer underlying the outer layer and an adhesive underlying the outer layer. The adhesive layer forms an annular adhesive surface wherein the dermal treatment device is configured to form a cavity for receiving the dermal clearing agent between an inner surface of the dermal treatment device and a dermal surface when the annular adhesive surface is attached to the dermal surface. The device also includes a fluid conduit operable to provide fluid access to the cavity through the outer layer and the reinforcement layer.

In another exemplary embodiment, a method of reducing photon scatter within a dermal tissue includes applying an annular adhesive surface of a dermal treatment device over an area of the dermal tissue. The dermal treatment device is configured to form a cavity between an inner surface of the dermal treatment device and the area when the annular adhesive surface is attached to the area. The adhesive underlies an outer layer. An outer layer is formed of a fluid impermeable polymer film, A reinforcement layer underlies the outer layer. A fluid conduit is operable to provide fluid access to the cavity through the outer layer and the reinforcement layer. The method further includes supplying a dermal clearing agent to the cavity.

In a further exemplary embodiment, a device includes a fluid impermeable polymer layer, a reinforcement material associated with the polymer layer, and an adhesive material configured to form an annular adhesive surface. The annular adhesive surface is configured to define a cavity between a tissue and the device when the annular adhesive surface is in contact with the tissue. The device further includes a fluid conduit configured to provide fluid access to the cavity through the polymer layer,

### BRIEF DESCRIPTION OF THE DRAWINGS

FIG. 1 and FIG. 2 include illustrations of an exemplary dermal treatment device when placed in contact with a treatment area.

FIG. 3, FIG. 4, FIG. 5, FIG. 6, and FIG. 7 include illustrations of exemplary dermal treatment devices.

FIG. 8 includes an illustration of an exemplary dermal treatment device when placed in contact with a treatment area.

FIG. 9 includes an illustration of an exemplary dermal treatment system.

FIG. 10 includes an illustration of an exemplary dermal treatment kit.

FIG. 11 includes an illustration of an exemplary method to treat a dermal tissue.

FIG. 12 includes an illustration of an exemplary dermal treatment device.

### DETAILED DESCRIPTION OF THE DRAWINGS

In a particular embodiment, a dermal treatment device includes an outer layer, a reinforcement layer, an adhesive, and a fluid conduit. The outer layer is formed of a fluid-impermeable polymer film and forms an outer major surface of the dermal treatment device. The reinforcement layer underlies the outer layer such that it is between the outer layer and a dermal surface when the dermal treatment device is attached to the dermal surface. An adhesive underlying the outer layer forms an annular adhesive surface. The dermal treatment device is configured to form a cavity between an inner surface of the dermal treatment device and a dermal surface when the annular adhesive surface is attached to the dermal surface. The fluid conduit provides fluid access to the cavity through the outer layer and the reinforcement layer. The dermal treatment device also may include an inner liner underlying the reinforcement layer and forming an inner major surface of the dermal treatment device.

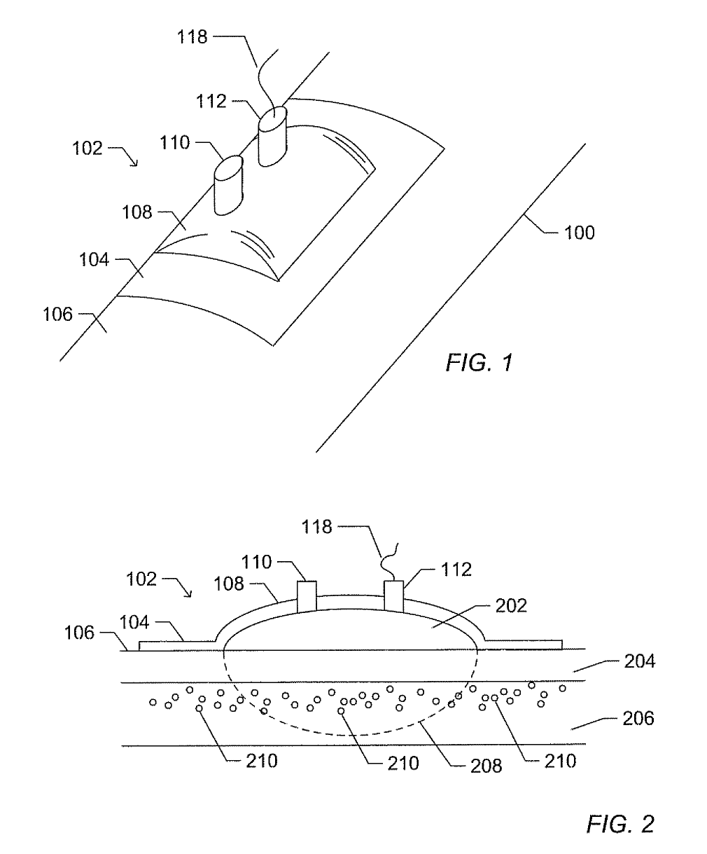

FIG. 1 and FIG. 2 include illustrations of an exemplary dermal treatment device 102 adhered to or attached to a dermal tissue 106. The dermal treatment device 102 may include an outer annular area 104 having an adhesive surface that attaches to a surface of the dermal tissue 106. In addition, the treatment device 102 includes an unattached region 108. The unattached region 108 is configured to form a cavity 202 between the dermal treatment device 102 and the dermal tissue 106 when a dermal clearing agent is placed within the cavity 202.

In an example, the dermal treatment device 102 may include a fluid conduit 110 configured to receive fluid and transfer the fluid into and out of the cavity 202. The fluid conduit 110 may include a luer adaptor and port to which a syringe or a tube connector may be attached. In another example, the fluid conduit 100 may include a check valve.

The dermal clearing agent may include an agent having a refractive index within about 20% of the refractive index of collagen. In a particular example, collagen has a refractive index of about 1.5. The dermal clearing agent may have a refractive index of between 1.4 and 1.6. For example, the dermal clearing agent may include a hyperosmotic agent, which when introduced into the skin tends to drive water away from the intracellular space. An exemplary dermal clearing agent may include glycerol, dimethylsulfoxide (DMSO), high-concentration dextrose solution, diatrizoate meglumine acid, or any combination thereof. In a further example, the dermal clearing agent also may include an anesthetic agent, an antiseptic agent, an antibacterial agent, an osmotic agent, a skin permeation enhancing agent, an ionic agent, or any combination thereof. For example, the dermal clearing agent may include an anesthetic agent, such as lidocaine. An exemplary skin permeation enhancing agent may include sodium lauryl sulfate or Azone (Nealson Research and Development, Irvine, Calif.).

When the cavity 202 includes a dermal clearing agent, the dermal clearing agent may be transferred into a region 208 of the dermal tissue 106. In particular, the treated region 208 may extend through the epidermis 204 and into the dermis 206. In a particular example, when using laser treatment, the tissue to be treated may reside at or below the dermis 206. For example, tissue 210 containing tattoo pigment may reside at the dermis 206 near the dermis-epidermis interface between the epidermis 204 and the dermis 206. As such, the dermal clearing agent may be transferred into a region 208 that includes the epidermis above the tissue 210 to be treated and may include the dermis 206 surrounding the tissue 210.

In addition, the dermal treatment device 102 may include an instrument 112 connected to other instrumentation via link 118. For example, the instrument 112 may include a sensor. An exemplary sensor includes a pressure sensor, an optical sensor, an electrolyte sensor, or any combination thereof. In another example, the instrument 112 may include a voltage source, a heat source, an ultrasonic transducer, or any combination thereof.

After a period of treatment, the dermal treatment device is typically removed, exposing a clarified dermal tissue 106. In an exemplary embodiment, laser treatments performed through the clarified dermal tissue 106 can be more effective at treating tissue 210.

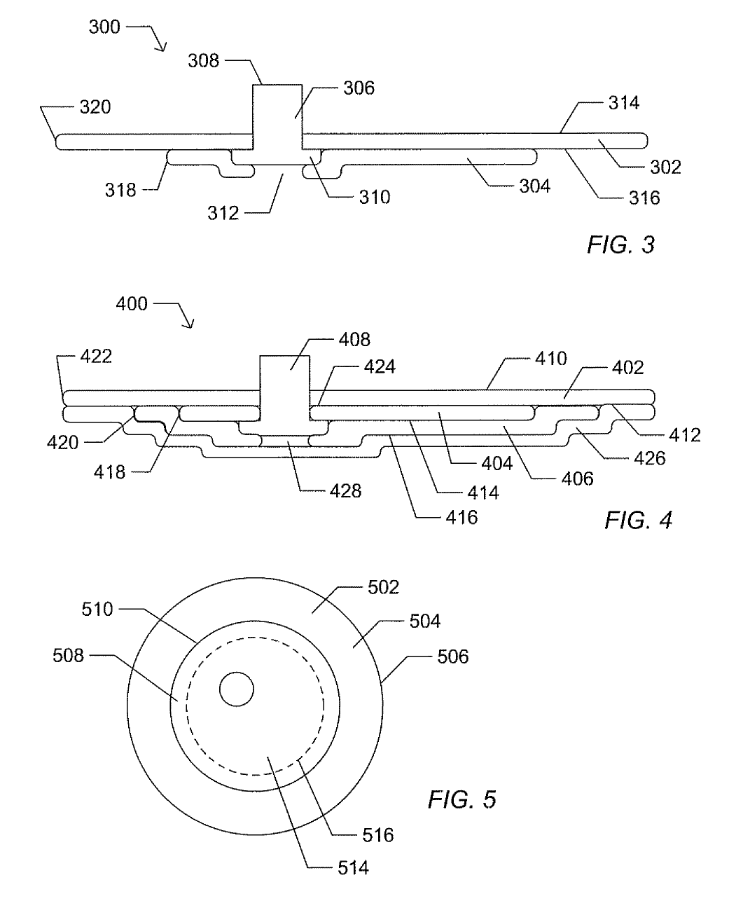

FIG. 3 includes an illustration of an exemplary device 300 that includes an outer layer 302 and a reinforcement layer 304. In addition, the dermal treatment device 300 includes a fluid conduit 306.

In an exemplary embodiment, the outer layer 302 is formed of a fluid-impermeable polymeric film. For example, the outer layer 302 may include one or more layers of polymeric or composite materials. In a particular example, the outer layer 302 includes a polymeric film, such as a polyolefin, a polyurethane, an acrylic polymer, a halogenated polyolefin, a silicone, or any combination thereof. In a particular example, the outer layer may include an olefinic elastomer. In another example, the outer layer may include polyethylene. In a further example, the outer layer may include a polyurethane film.

In an exemplary embodiment, the outer layer 302 is configured to form a major surface 314 of the dermal treatment device 300. In addition, the outer layer 302 may include an adhesive surface 316. In an example, the adhesive surface 316 of the outer layer 302 includes an adhesive. For example, the adhesive may include a pressure-sensitive adhesive, such as an acrylic pressure-sensitive adhesive. In an example, the adhesive is a medical-grade pressure-sensitive acrylic adhesive. In a particular example, the reinforcement layer 304 is adhered to the adhesive surface 316 of the outer layer 302. In an alternative embodiment an adhesive may be applied separately from the dermal treatment device.

The reinforcement layer 304 may be formed of a polymeric material. For example, the reinforcement layer 304 may be formed of a polyolefin, a vinyl polymer, a polyester, or any combination thereof. In a particular example, the reinforcement layer 304 may be a polymeric sheet. Alternatively, the reinforcement layer 304 may be a woven or random fibrous material. While the reinforcement is illustrated as a reinforcement layer 304, reinforcement alternatively may be incorporated into the outer layer 302.

In a particular embodiment, the reinforcement layer 304 is configured to provide a desirable stiffness and modulus to the dermal treatment device 300. In a particular example, the reinforcement layer 304 may be thicker than the outer layer 302. For example, the outer layer 302 may have a thickness in a range between about 1 mil (25.4 microns) and about 10 mils (254 microns), such as a thickness in a range between about 2 mils (50.8 microns) and about 5 mils (127 microns). The reinforcement layer 304 may have a thickness in a range between about 2 mils (50.8 microns) and about 15 mils (381 microns), such as a range between about 4 mils (101.6 microns) and about 10 mils (254 microns).

In another exemplary embodiment, the outer layer 302 may have an elastic modulus not greater than the elastic modulus of the reinforcement layer 304. For example, the elastic modulus of the reinforcement layer 304 may be at least about 400 ksi (2.75 GPa), such as at least about 500 ksi (3.44 GPa), or even, at least about 600 ksi (4.13 GPa).

In a further exemplary embodiment, the outer layer 302 may have an elongation greater than an elongation of the reinforcement layer 304. For example, the outer layer 302 may have an elongation of at least about 80%, such as at least about 100%, or even at least about 150%. The reinforcement layer 304 may have an elongation not greater than about 200%, such as an elongation not greater than about 100%, or even, not greater than about 60%. In a particular example, the reinforcement layer 304 may have an elongation not greater than about 40%.

In a further example, the outer layer 302 has a contour 320 that extends horizontally beyond an outer contour 318 of the reinforcement layer 304. As such, the adhesive layer 316 is exposed in an annular region to form an annular adhesive surface beyond the horizontal contour 318 of the reinforcement layer 304. As such, when the annular adhesive surface formed by the adhesive surface 316 of the outer layer 302 is placed in contact with a dermal tissue, the region between the reinforcement layer 304 and the dermal tissue may be used to form a cavity in which a dermal clearing agent may be placed. Alternatively, a portion of the reinforcement layer 304 may include adhesive such that the annular adhesive surface includes part of the surface of the reinforcement layer 304.

In addition, the dermal treatment device 300 may include a fluid conduit 306. For example, the fluid conduit 306 may include a port 308 configured to attach to a syringe or a tubing attachment. In addition, the fluid conduit 306 may include a valve, such as a check valve. Further, the fluid conduit 306 may include a mechanism to secure the fluid conduit 306 in place within the dermal treatment device 300. For example, the fluid conduit 306 may include a flange 310. In a particular example, the fluid conduit 306 may include a luer adapter. In the illustrated embodiment, the flange 310 is placed between the outer layer 302 and the reinforcement layer 304. The reinforcement layer 304 includes a hole 312 to permit fluid transfer between the fluid conduit 306 and a cavity formed between the reinforcement layer 304 and a dermal tissue. Alternatively, the flange can be placed between any two layers as long as a hole or passage extends through each of the layers.

FIG. 4 includes an illustration of a further exemplary embodiment of a dermal treatment device 400. The dermal treatment device 400, for example, includes an outer layer 402, a reinforcement layer 404, and a liner 406. In addition, the dermal treatment device 400 may include a fluid conduit 408 and a release liner 426.

In an example, the outer layer 402 forms a major surface 410 of the dermal treatment device 400. In addition, the outer layer 402 may include an adhesive surface 412. In a particular example, the outer layer 402 is formed of a fluid-impermeable polymeric films.

As illustrated, the reinforcement layer 404 underlies the outer layer 402. In a particular example, the reinforcement layer 404 is formed of a polymeric sheet that adheres to the adhesive surface 412 of the outer layer 402.

The liner 406 underlies the reinforcement layer 404. In an example, the liner 406 may include an adhesive surface 414. As such, the liner 406 may adhere to the reinforcement layer 404 and a portion of the outer layer 402. In addition, the liner 406 may include a non-adhesive surface 416.

In an example, the liner 406 may be formed of a polymeric film. In a particular example, the polymeric film may be a fluid-impermeable polymeric film. For example, the liner 406 may include a polymeric material, such as a polyolefin, a polyurethane, a halogenated polyolefin, a silicone, or any combination thereof. In particular, the liner 406 may be formed of a material similar to the material forming the outer layer 402.

In addition, the dermal treatment device 400 may include a fluid conduit 408. The fluid conduit 408, for example, may be a flanged luer adapter, including flange 424 located between the reinforcement layer 404 and the liner 406. Alternatively, the flange 424 may be located between the reinforcement layer 404 and the outer layer 402.

The liner 406 may include a hole 428 to permit a dermal clearing agent to be transferred between the fluid conduit 408 and a cavity formed between a dermal tissue and the dermal treatment device 400. The dermal clearing agent may include an agent having a refractive index within about 20% of the refractive index of collagen. In a particular example, collagen has a refractive index of about 1.5. The dermal clearing agent may have a refractive index of between 1.4 and 1.6. For example, the dermal clearing agent may include a hyperosmotic agent, which when introduced into the skin tends to drive water away from the intracellular space. An exemplary dermal clearing agent may include glycerol, dimethylsulfoxide (DMSO), high-concentration dextrose solution, diatrizoate meglumine acid, or any combination thereof. In a further example, the dermal clearing agent also may include an anesthetic agent, an antiseptic agent, an antibacterial agent, an osmotic agent, a skin permeation enhancing agent, an ionic agent, or any combination thereof. For example, the dermal clearing agent may include an anesthetic agent, such as lidocaine. An exemplary skin permeation enhancing agent may include sodium lauryl sulfate or Azone (Nealson Research and Development, Irvine, Calif.).

In a further exemplary embodiment, the dermal treatment device 400 may include a release liner 426. The release liner 426 may be configured for removal prior to adhesion of the dermal treatment device 400 to a dermal tissue. The release liner 426, for example, may be formed of a treated paper, cloth, polymeric film, or any combination thereof. In a particular example, the liner 426 may be formed of a silicone-treated paper or cloth. In another example, the release liner 426 may be formed of a silicone film, halogenated polyolefin, or any combination thereof.

In a particular example, the outer contour 420 of the liner 406 extends horizontally beyond the outer contour 418 of the reinforcement layer 404. As such, a portion of the liner 406 may extend to contact a portion of the outer layer 402. The outer layer 402 may include an outer contour 422 that extends horizontally beyond the outer contour 420 of the liner 406. In particular, the adhesive surface 412 of the outer layer 402 may form an annular adhesive surface extending from the outer contour 420 of the liner 406 to the outer contour 422 of the outer layer 402. Alternatively, the adhesive surface may be formed by an adhesive layer or glue applied in an annular pattern around an inner major surface of the dermal treatment device 400 such that, for example, a portion of the underlying surface of the liner 406 may form a part of the annular adhesive surface. In general, the width of the annular surface extending between the contours 420 and 418 varies in accordance with the size of the dermal treatment device 400 and the intended operating conditions. In a particular example, the width is at least about 0.39 in (1 cm), such as at least about 0.78 in (2 cm).

FIG. 5 further illustrates an annular adhesive surface from a plan view. For example, an outer contour 510 of a liner 508 may extend radially beyond an outer contour 516 of a reinforcement layer 514. The outer contour 506 of the outer layer 502 may extend radially beyond the outer contour 510 of the liner 508. As a result, an annular adhesive surface 504 is formed from an adhesive surface of the outer layer 502. In addition, a fluid conduit or other instrumentation 512 may extend through each of the layers.

While FIG. 5 includes an illustration of the dermal treatment device in a circular form, the dermal treatment device may form a square, an oval, a polygon, an irregular shape, or any combination thereof. As such, the annular adhesive surface may form a shape, such as a circular, polygonal, or irregular shape, or any combination thereof. As illustrated in FIG. 6, the reinforcement layer 606, liner 604, and an outer layer 602 may be formed in a polygonal shape, such as a rectangle. As such, the annular adhesive surface 610 extends in a rectangular shape surrounding the liner 604. In a further example illustrated in FIG. 7, a reinforcement layer 706, a liner 704, and an outer layer 702 are in the form of an oval. As such, the annular adhesive surface 710 takes an ovular shape surrounding the liner 704.

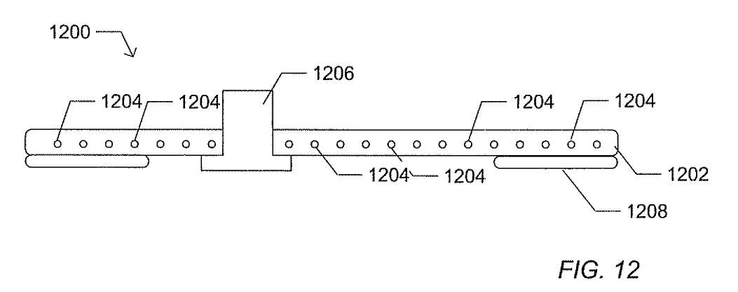

In an alternative embodiment of the dermal treatment device illustrated in FIG. 12, a reinforcement material 1204 may be associated with a fluid-impermeable polymer film 1202. For example, when the reinforcement material 1204 is a fibrous material, the reinforcement material can be incorporated within the fluid impermeable polymer film 1202. In particular, the reinforcement material 1204 may include a woven fibrous material. In addition, an adhesive 1208 may form an annular adhesive surface. Further, a fluid conduit 1206 may provide access, through the fluid-impermeable film 1202 and the reinforcement material 1204, to a cavity defined by the annular adhesive surface when the adhesive 1208 is in contact with a tissue.

In a particular embodiment, the dermal treatment device, excluding the fluid conduit and sensor, may have a thickness in a range between about 1 mil (25.4 microns) and about 20 mils (508 microns), such as a thickness in a range between about 5 mils (127 microns) and about 10 mils (254 microns). In addition, the elastic modulus of dermal treatment device may be at least about 400 ksi (2.75 GPa), such as at least about 500 ksi (3.44 GPa), or even, at least about 600 ksi (4.13 GPa). Further, the dermal treatment device may have an elongation not greater than about 200%, such as an elongation not greater than about 100%, or even, not greater than about 60%. In a particular example, the dermal treatment device may have an elongation not greater than about 40%.

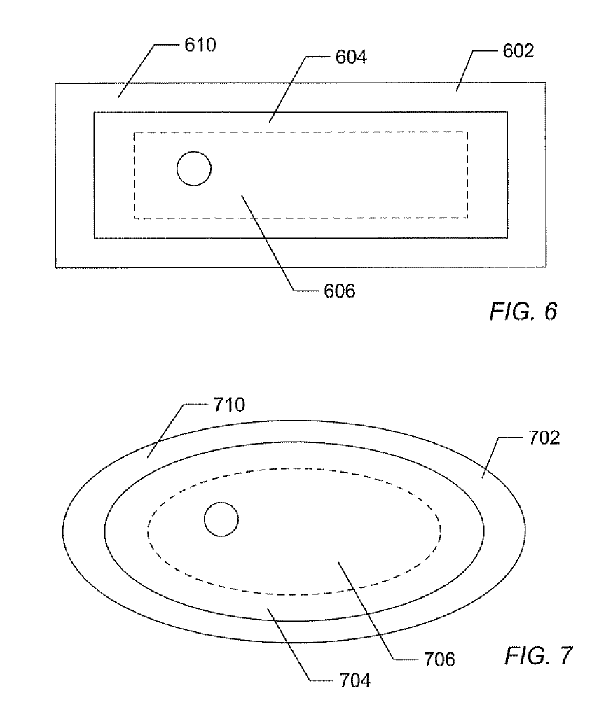

Returning to FIG. 4, before the dermal treatment device is placed in contact with a dermal tissue, the release liner 426 may be removed to expose the annular adhesive surface 412. The annular adhesive surface 412 may adhere to a dermal tissue. As a result, a cavity may be formed between the dermal tissue and the liner 406. For example, FIG. 8 includes an illustration of an exemplary dermal treatment device 800. The dermal treatment device 800 may include an outer layer 802, a reinforcement layer 804, a liner 806, and a fluid conduit 810 extending through the outer layer 802 and the reinforcement layer 804. In addition, the dermal treatment device 800 may include an instrument 812. For example, the instrument 812 may be a sensor, such as a pressure sensor, an optical sensor, an electrolyte sensor, or any combination thereof. In another example, the instrument 812 may include an activation device, such as a voltage source, a heater, a sonic transducer, or any combination thereof.

In particular, an annular adhesive surface 816 bonds to a portion of the surface 818 of a dermal tissue. The liner 806 forms an inner major surface 820 of the dermal treatment device 800 that is non-adhesive. As a result, a cavity 814 is formed between the inner major surface 820 and the surface 818 of the dermal tissue. In a particular example, a dermal clearing agent may be supplied to the cavity 814 via the fluid conduit 810. In particular, the dermal clearing agent may be pressurized to further influence transfer of the dermal clearing agent into the dermal tissue.

In an exemplary embodiment, the dermal treatment device 800 provides clearing in a reduced time. To measure the effectiveness of clearing agent administration, a Relative Clearing Index can be defined based on measurement of the reflectance spectrum for the skin. For example, the reflectance spectrum of human skin when recorded as A=log(R₀/R) exhibits a broad relatively featureless rise in the near-infrared region. This apparent nearly-linear increase in absorbance is due to the fact that, as wavelength increases, scattering coefficient decreases and less light is returned. As scattering is reduced by index matching or another mechanism, the effect on the reflectance spectrum can be more pronounced at shorter wavelengths where scattering is typically higher. Thus, the slope of the reflectance spectrum may appear diminished in magnitude. Generally, reduced scatter at shorter wavelengths means less light is scattered reflectively and apparent absorption goes up. As such, a linear regression of log(R₀/R) spectral data against wavelength for a suitable portion of the spectrum (i.e., over the wavelength range between about 650 nm and about 750 nm) can yield slope coefficients whose relative magnitudes are indicative of the amount of clearing observed. Specifically, for testing in guinea pigs, the slope coefficients may change from 3.0 prior to treatment to 1.5 post treatment. A Relative Clearing Index is defined as the ratio of the change in coefficient to the initial coefficient. As such, a Relative Clearing Index for a guinea pig model is 0.5, ((3.0−1.5)/3.0=0.5). In human studies, the Relative Clearing Index may be at least about 0.1, such as at least about 0.12, or even about 0.16

In a further example, the dermal treatment device 800 may exhibit a desirable Clearing Time, defined as the period of treatment at which the Relative Clearing Index exceeds 0.15 in a human model. For example, the Clearing Time may be not greater than about 1 hour 30 minutes, such as not greater than about 1 hour, or even, not greater than about 45 minutes.

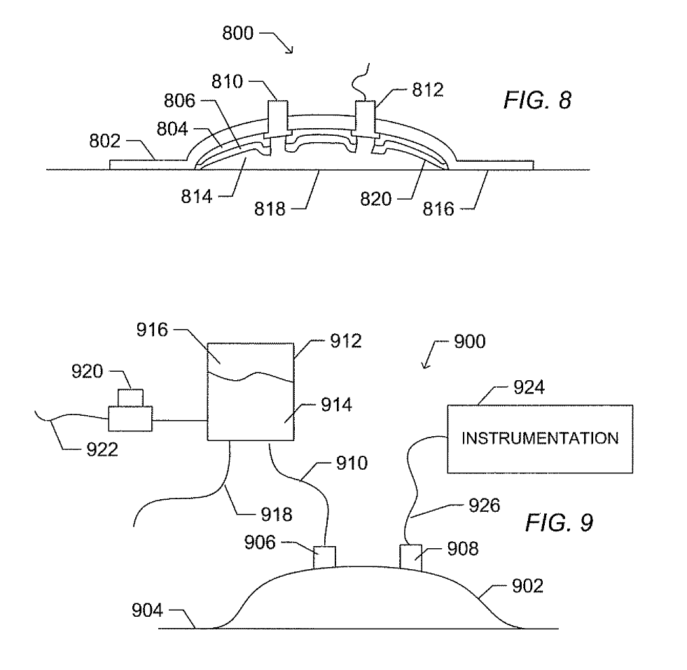

Using the dermal treatment device, a patient may be treated to clarify a portion of the dermal tissue for further laser treatment. For example, FIG. 9 includes an illustration of an exemplary system 900 for clarifying dermal tissue and reducing photon scatter. The exemplary system 900 may include a dermal treatment device 902 to be adhered to a dermal tissue 904. In a particular example, the dermal treatment device 902 may include a fluid conduit 906 and an instrument 908. The fluid conduit 906 may be coupled to a hose or tube 910 configured to supply pressurized dermal clearing agent to the dermal treatment device 902

The tubing 910, for example, may be coupled with a pressurized container 912 including the dermal clearing agent 914. For example, the pressurized container 912 may be pressurized with air or an inert gas, such as nitrogen or helium. In particular, the air or inert gas may form a blanket 916 that applies pressure to the dermal clearing agent, forcing the dermal clearing agent through tube 910 and subsequently, to the cavity of the dermal treatment device 902. For example, the blanket 916 of gas may be provided through a supply line 922 that extends through a pressure regulator 920. In an exemplary embodiment, the pressure of the gas may be regulated to a pressure and a range between about 0.1 psi and about 10 psi. In a particular example, the pressure of the gas is at least about 0.5 psi, such as at least about 3 psi. As a result, the pressure in the cavity of the dermal treatment device 902 may be at least about 0.5 psi, such as at least about 3 psi. The container 912 also may include a dermal clearing agent supply line 918.

In addition, the dermal treatment device 902 may include an instrument 908. The instrument 908 may be attached to additional instrumentation 924 via communication link 926. In an exemplary embodiment, the instrument 908 may be a sensor, such as a pressure sensor, an optical sensor, an electrolyte sensor, or any combination thereof. The instrumentation 924 may be configured to record or display data acquired from a sensor. In another exemplary embodiment, the instrument 908 may include an activation device. For example, an activation device may be configured to enhance transfer of dermal clearing agent into a dermal tissue. In a particular example, the activation device includes a voltage source, a heater, a sonic transducer, or any combination thereof. For example, the instrumentation 924 may be configured to operate the activation device.

In a particular embodiment, the instrument 908 is a sensor, such as a pressure transducer. The sensor may provide pressure data to the instrumentation 924. A feedback loop (not shown) may activate the valve 920 to influence the pressure within the chamber 912 to control the pressure within a cavity of the dermal treatment device 902. Alternatively, the instrumentation may control pressure oscillations within the cavity to provide a pulsating force to drive agent into the dermal tissue. In another exemplary embodiment, the instrument 908 may be a sensor, such as an optical transducer. The optical transducer may provide data indicating when to end treatment of the dermal tissue.

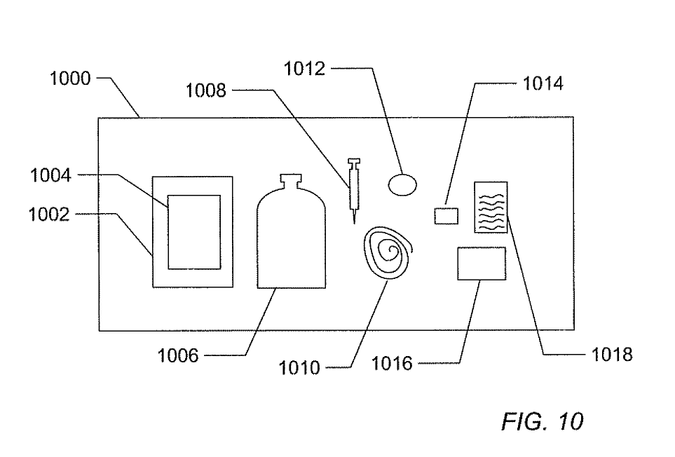

The dermal treatment system or portions thereof may be provided in a dermal treatment kit FIG. 10 includes an illustration of an exemplary kit 1000. The exemplary kit 1000 includes a dermal treatment device 1004. The dermal treatment device 1004 may be included in a sealed packet 1002. In addition, the kit 1000 may include a dermal clearing agent 1006. For example, the dermal clearing agent may include a hyperosmotic agent, which when introduced into the skin tends to drive water away from the intracellular space. An exemplary dermal clearing agent may include a hyperosmotic agent such as glycerol, dimethylsulfoxide (DMSO), high concentration dextrose solution, diatrizoate meglumine acid, or any combination thereof. In a particular example, the dermal clearing agent includes between about 75% and about 100% of a hyperosmotic agent. In a further example, the dermal clearing agent also may include an anesthetic agent, an antiseptic agent, an antibacterial agent, an osmotic agent, a skin permeation enhancing agent, an ionic agent, or any combination thereof. For example, the dermal clearing agent may include an anesthetic agent, such as lidocaine. An exemplary skin permeation enhancing agent may include sodium lauryl sulfate or Azone (Nealson Research and Development, Irvine, CA).

Further, the kit 1000 may include a supply tool, such as a hose system 1010 or a syringe 1008 configured to deliver the dermal clearing agent 1006 to the dermal treatment device 1004. In addition, the kit 1000 may include an instrument 1014 for attachment to the dermal treatment device 1004 or instrumentation 1016 to attach to the instrument 1014.

Often, a dermal tissue is prepared prior to attaching or adhering the dermal treatment device 1004 to a surface of the dermal tissue. As such, the kit 11000 may include topical ointments or abrasives to remove the surface of the skin, such as the stratum corneum. In a particular example, the kit 1000 includes a stripping tape 1012 useful for removing the stratum corneum. In another example, the kit 1000 can include an instant cure adhesive, such as a cyano acrylate, useful in glue stripping of a dermal surface. In a further example, the kit 1000 may include a skin permeation enhancer, such as sodium lauryl sulfate and Azone (Nelson Research and Development, Irvine, Calif.). In an additional example, the kit 1000 may include lotions containing microstructures that embed in the stratum corneum and facilitate its removal, dermabrasive topical ointments and devices, chemical peels, or any combination thereof. Further the kit 1000 may include a separate adhesive.

In addition, the kit 1000 may include written instructions 1018 indicating methods of using the various components of the kit 1000. The instructions 1018 may direct the use of the dermal treatment device 1004, the assembly of the dermal treatment system that includes the dermal treatment device 1004, counter-indications for use of such devices, warnings and caveats, or any combination thereof.

In an example, the kit 1000 may be included in a single container and sealed. Alternatively, the kit 1000 may be an assembly of various containers including various components of the kit 1000.

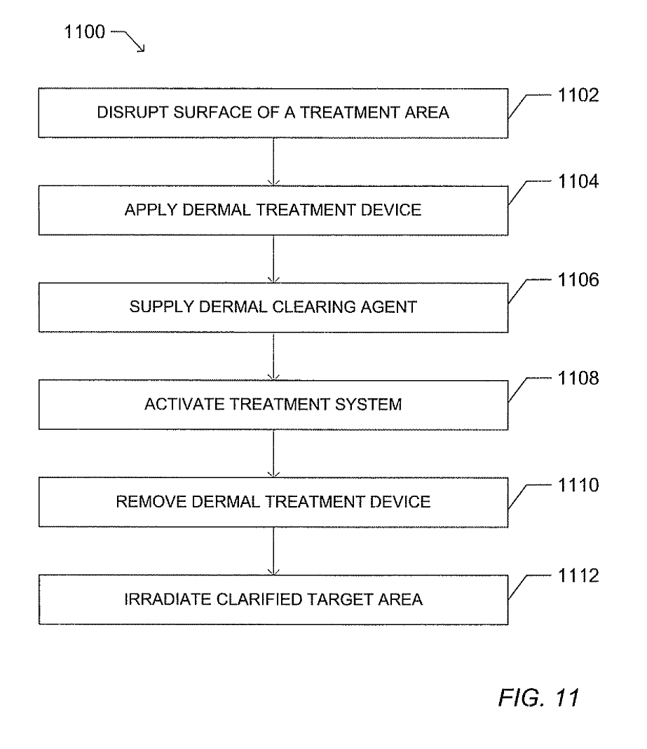

In an exemplary embodiment, the dermal treatment device may be used to reduce photon scanner within a dermal tissue or to alter the bulk refractive index of the tissue. FIG. 11 includes an illustration of an exemplary method 1100 for treatment of a dermal tissues. For example, a surface of the treatment area of the dermal tissue, such as the stratum corneum within the treatment area, may be removed, disrupted, or compromised, as illustrated at 1102. For example, the stratum corneum may be tape-stripped using an adhesive tape that is repetitively adhered to and removed from the dermal tissue. Alternatively, the surface of the dermal tissue may be treated using dermabrasion, microdermabrasion, sonication, heating, laser ablation, microporation, chemical delipation, or any combination thereof. For example, topical ointments or abrasive ointments may be used to abrade or dissolve the stratum corneum. In another example, an ionic or polarizing component may be used in conjunction with iontophoretic current to increase permeability through the stratum corneum. Finely focused lasers may be used to create micro-pores in the stratum corneum. For example, a Erbium:Yttrium-Aluminum-Garnet (Er:YAG) or a near infra red (NIR) diode and an absorbing ink may be used to disrupt the stratum corneum. Heat may be applied to increase mass transport and enhance permeability of the stratum corneum. In addition, heat or steam may facilitate removal of the stratum corneum. In a further example, a skin permeation enhancer, such as sodium lauryl sulfate, Azone (Nelson Research and Development, Irvine, Calif.), or any combination thereof, may be used to improve skin permeation. In another example, the dermal tissue may be resurfaced using lasers, such as a CO₂ or Er:YAG laser, to remove the stratum corneum. The stratum corneum also may be removed using adhesive glue to glue strip the stratum corneum from the surface of the dermal tissue. Steam, lotions, or other activators may be used to enhance adhesion between tape or glue and skin. Further, photomechanical methods may be used to drive large molecules through the stratum corneum to create access channels. In addition, dermabrasive techniques may be used. In a further example, over-tattooing or maceration of an agent into the tissue may be used to provide a path through the stratum corneum.

The dermal treatment device may be applied to the dermal tissue or a surface thereof, as illustrated at 1104. For example, a release liner of the dermal treatment device may be removed from the dermal treatment device exposing an annular adhesive surface. The annular adhesive surface may be adhered to a portion of the surface of the dermal tissue. In an example, the dermal treatment device is applied to the skin at least about 1 minute prior to injecting an agent to permit improved adhesion to the surface.

Optionally, a dehydrating agent may be applied within the cavity for a period prior to applying the dermal clearing agent. In an example, the dehydrating agent may be injected into a cavity formed between the dermal treatment device and the surface of the dermal tissue. Alternatively, the dehydrating agent may be applied to the surface prior to applying the dermal treatment device.

A dermal clearing agent may be supplied to a cavity formed by the dermal treatment device and the surface of the dermal tissue, as illustrated at 1106. For example, the dermal clearing agent may include a hyperosmotic agent, which when introduced into the skin tends to drive water away from the intracellular space. An exemplary hyperosmotic agent may include glycerol, dimethylsulfoxide (DMSO), high-concentration dextrose solution, diatrizoate meglumine acid, or any combination thereof. In an example, the dermal clearing agent includes between about 75% and about 100% of the hyperosmotic agent. In a further example, the dermal clearing agent also may include an anesthetic agent, an antiseptic agent, an antibacterial agent, an osmotic agent, a skin permeation enhancing agent, an ionic agent, a hyperemic agent, or any combination thereof. For example, the dermal clearing agent may include an anesthetic agent, such as lidocaine. An exemplary skin permeation enhancing agent may include sodium lauryl sulfate or Azone (Nealson Research and Development, Irvine, Calif.).

In a particular example, the dermal clearing agent is provided to the cavity at a particular pressure. For example, the dermal clearing agent may be provided at a pressure of at least about 0.5 psi, such as at least about 3 psi.

In a particular embodiment, a treatment system may be activated, as illustrated at 1108. For example, the pressure may be modulated to apply a pulsating force to the tissues. In another example, heat may be applied to increase perfusion and mass transport of the agent into the tissue. In a further example, the dermal clearing agent may include ions and electrical current may be used to force agent into the tissue, e.g., through electrophoresis or iontophoresis. In a particular example, the dermal treatment device includes a voltage source that can result in a voltage difference between the voltage source and a grounded dermal tissue or a dermal tissue activated to have a different voltage. In another exemplary embodiment, the dermal treatment device may include an ultrasonic device that provides ultrasonic waves directed at the surface of the dermal tissue. Such ultrasonic or low intensity ultrasound vibrations may be used to mechanically force agent into the tissue.

After a period of treatment, the dermal treatment device may be removed, as illustrated at 1110. For example, a period of treatment may be set by previous experimentation. In an example, the dermal treatment device may be left in place for a period extending at least about 5 minutes, such as at least about 15 minutes or at least about 30 minutes. In another exemplary embodiment, the dermal treatment device may include a sensor, such as a pressure sensor, an optical sensor; or an electrolytic sensor. In an exemplary embodiment, a pressure may be measured to determine an amount of dermal clearing agent transferred into a dermal tissue. In another exemplary embodiment, an optical sensor may be used to determine the clarity or photonic scatter within a dermal tissue. In a further exemplary embodiment, an electrolytic sensor or a sensor of analytes may be used to determine a condition of the tissue based on analytes within the solution above the tissue. Once a measurement approaches a desired value, a dermal treatment device may be removed.

As a result of the use of the dermal treatment device, the index of refraction or the photon scattering of the dermal tissue may be altered. Subsequently, the clarified dermal tissue may be treated using radiation, such as electromagnetic radiation, including laser treatment. In particular, a laser treatment may be used to preferentially degrade or treat tissue having aspects that absorb the laser radiation preferentially. For example, ink particles of a tattoo may preferentially absorb particular wavelengths of electromagnetic radiation relative to surrounding tissue.

Further treatment of the tissue may be used to prevent tissue necrosis. For example, heat maybe applied to increase perfusion and removal of agents. In another example, a dermal treatment device may be used to irrigate the affected site with chemical washes to remove agent from the tissue or apply an iso-osmotic agent or diluent to the affected site. In a further example, a reverse iontophoretic current may be applied to the tissue to extract agents. In addition, the tissue further may be treated by application of Neosporin, lotion, petroleum jelly, moist gauze, or any combination thereof.

Particular embodiments of the dermal treatment device advantageously improve light scatter within dermal tissue. In particular, the dermal treatment device can provide faster dermal clearing or altering of index of refraction than traditional injection methods. In addition, particular embodiments of the dermal treatment device advantageously provide quick dermal clearing with reduced tissue necrosis. In particular, the dermal treatment device may cause clarification of the dermal tissue while limiting later necrosis in the tissue. For example, treated tissue may have no observable necrosis 72 hours post treatment. In addition, a reduced amount of light scattering in the dermal tissue after treatment may permit use of shorter-wavelength laser treatments more effective in treating longer wavelength-colored tattoos (e.g., red, orange, or yellow).

### EXAMPLES

### Example 1

### Device Assembly

A 0.25 inch hole is cut in the center of two bandages (a 4″×4.75″ Tegaderm™ Hp bandage and a 2″×2.75″ Tegaderm™ bandage, available from 3M). A release paper backing of the Tegaderm™ HP bandage is pealed, exposing an adhesive side of the Tegaderm™ HP bandage. A hole in the Tegaderm™ HP bandage is aligned with a hole in a cutting board and a luer adapter is inserted in the hole, the luer-end first A flat surface of the adapter bonds to the adhesive of the Tegaderm™ HP bandage. A vinyl reinforcing layer is applied to the adhesive surface of the Tegaderm™ HP bandage, aligning a hole in the vinyl reinforcing layer with the end of the luer adapter. The 2″×2.75″ Tegaderm™ bandage is applied over the vinyl reinforcement layer with the adhesive surface contacting the vinyl reinforcement layer and overlapping the vinyl reinforcement layer to contact the adhesive surface of the Tegaderm™ HP bandage. The assembly is placed in a clean receivable bag for later use.

### Example 2

### Dermal Clearing

The dermal treatment area of a human subject is washed thoroughly with an antibacterial soap. All hair is removed from the target area. Hair may be removed by shaving the target area. The area is dried well with a clean cloth or paper towel.

The stratum corneum in the target area is disrupted. An adhesive tape is applied to the target area. The tape is allowed to rest for thirty seconds to one minute. The tape is peeled from the target area along the axis of the tape. The above tape stripping is repeated for at least thirty times over the same area. In general, tape-stripping should be repeated until the stratum corneum is removed as indicated by a glistening appearance of the target area. Alternatively, glue stripping is used. Super glue is applied to a clean glass slide. The slide is applied to the target area and pressure is maintained for approximately one minute and thirty seconds. The slide is allowed to rest for an additional minute to a minute thirty seconds. The slide is peeled from the target area along the axis of the slide. Glue stripping is repeated for at least three times over the same area.

The dermal treatment device is applied to the target area. The adhesive surface of the dermal treatment device is applied to the skin and allowed to rest for thirty to forty-five minutes to ensure sufficient adhesion to the skin. An injection port is installed on the luer adapter. A Touhy Borst adapter is installed over the needle as a stop to prevent inadvertent contact between the skin and a needle. Repeatedly using a 10 cc syringe fitted with a 16 G 1.5″ needle, 70 cc to 100 cc of dermal clearing agent is transferred into the cavity formed between the bandage and the target area. The target area is exposed to the dermal clearing agent for approximately 40 minutes.

After 40 minutes, the injection port is removed, allowing the dermal clearing agent to be expressed or withdrawn from the cavity of the dermal treatment device. The dermal treatment device is removed. Improvement in light penetration may occur for at least the first fifteen minutes, and may persist for approximately thirty minutes.

Once treatment is performed, the area is washed with antibacterial soap. A layer of bacitracin ointment is applied to prevent infection, and a bandage is placed over the treated areas. No tissue necrosis is observed during a 72 hour observation period.

### Example 3

### Dermal Clearing Performance

Target areas on a set of human subjects awe treated. The target area is washed with antibacterial soap, scrubbing the area thoroughly for approximately 30 seconds. Hair is removed from the target area with a standard disposable shaving razor using the suds from the antibacterial soap as lubricant. The area is lightly dried with a paper towel.

For each of the patients, the stratum corneum is removed from the target area using the glue stripping method. The glue stripping method is repeated 3-5 times until glistening was observed. Glistening is a result of trans-epithelial water loss due to the removal or disruption of the stratum corneum.

The dermal treatment device is applied over the target area and allowed to rest a minimum of 30 minutes. The clearing agent is administered into the device until a pressure of approximately 30 mm Hg (0.58 psi) is achieved. The pressure within the application device is maintained by a remote pressurization device. The target area is exposed to the pressurized glycerin for a minimum of 45 minutes, but not exceeding 60 minutes. The application device is drained and removed. Excess clearing agent is removed with a paper towel.

Reflectance measurements are taken from several locations to calculate the spectrum and the Relative Clearing Index (See TABLE 1). The average Relative Clearing Index for successful scatter reduction in human applications is approximately 0.12, and can be greater than 0.16. The average Clearing Time is not greater than 60 minutes, generally between about 45 minutes and about 60 minutes.

TABLE 1
Relative Clearing Index for Human Subjects

| | Initial | Final | Relative |
|---|---|---|---|
| Subject No. | Coefficient | Coefficient | Clearing Index |
| 1. | 0.0027 | 0.0025 | 0.0748 |
| 2. | 0.0030 | 0.0026 | 0.1309 |
| 3. | 0.0030 | 0.0025 | 0.1627 |

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. A device comprising:
an outer layer formed of a fluid impermeable polymer film, the outer layer forming an outer major surface of the device;
a reinforcement layer underlying the outer layer;
an adhesive underlying the outer layer, the adhesive forming an annular adhesive surface, wherein the device is configured to form a cavity between an inner surface of the device and a dermal surface when the annular adhesive surface is attached to the dermal surface; and
a fluid conduit operable to provide fluid access to the cavity through the outer layer and the reinforcement layer.

2. The device of claim 1, wherein the outer layer extends beyond the outer contour of the reinforcement layer.

3. The device of claim 1, wherein the outer layer includes an olefinic elastomer.

4. (canceled)

5. (canceled)

6. (canceled)

7. The device of claim 1, wherein the reinforcement layer includes vinyl polymer.

8. The device of claim 1, wherein the reinforcement layer includes a polyolefin.

9. The device of claim 1, wherein the reinforcement layer includes a woven fiber.

10. The device of claim 1, wherein the outer layer has a thickness not greater than a thickness of the reinforcement layer.

11. (canceled)

12. (canceled)

13. (canceled)

14. (canceled)

15. (canceled)

16. The device of claim 1, wherein the reinforcement layer has an elastic modulus of at least about 400 ksi.

17. (canceled)

18. The device of claim 1, wherein the outer layer has an elongation of at least about 80%.

19. (canceled)

20. The device of claim 1, wherein the reinforcement layer has an elongation not greater than about 200%.

21. (canceled)

22. (canceled)

23. The device of claim 1, further comprising a liner underlying the reinforcement layer.

24. The device of claim 23, wherein the liner extends beyond the outer contour of the reinforcement layer and wherein the outer layer extends beyond the outer contour of the liner.

25. The device of claim 23, wherein the liner includes an adhesive surface in contact with the reinforcement layer.

26. (canceled)

27. The device of claim 1, wherein the adhesive is included on a major surface of the outer layer opposite the outer major surface.

28. (canceled)

29. (canceled)

30. The device of claim 1, further comprising a sensor operably coupled to the cavity.

31. (canceled)

32. (canceled)

33. (canceled)

34. (canceled)

35. A device comprising:
a fluid impermeable film;
a reinforcement material underlying the fluid impermeable film;
an adhesive forming at least an annular adhesive surface, wherein the device is configured to form a cavity between an inner surface of the device and a dermal surface when the annular adhesive surface is attached to the dermal surface; and
a fluid conduit configured to provide fluid access to the cavity through the fluid impermeable film and the reinforcement material.

36. The device of claim 35, further comprising a liner underlying the reinforcement material.

37. The device of claim 36, wherein the liner extends beyond an outer contour of the reinforcement material and wherein the fluid impermeable film extends beyond an outer contour of the reinforcement material.

38. (canceled)

39. (canceled)

40. (canceled)

41. (canceled)

42. (canceled)

43. (canceled)

44. A device comprising:
a fluid impermeable film having an outer major surface and an inner major surface, the inner major surface including an adhesive;
a reinforcement material underlying the fluid impermeable film, the reinforcement material having a elastic modulus greater than the elastic modulus of the fluid impermeable film; and
a liner underlying the reinforcement material, an outer contour of the liner extending beyond an outer contour of the reinforcement material, an outer contour of the fluid impermeable film extending beyond the outer contour of the liner the liner including an adhesive surface adhered to the reinforcement material and a portion of the fluid impermeable film and a non-adhesive surface configured to form a cavity between a dermal surface and the liner when the device engages the dermal surface.

45. The device of claim 44, further comprising a sensor operably coupled to the cavity.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)
